(19) United States
(12) Patent Application Publication
(10) Pub. No.: US 2020/0283472 A1
(43) Pub. Date: Sep. 10, 2020

(54) A PROCESS FOR PURIFICATION OF FC-FUSION PROTEINS

(71) Applicant: NAVYA BIOLOGICALS PVT. LTD, Hubli (IN)

(72) Inventor: RAJYASHRI RAMAKRISHNAN KARUR, HUBLI (IN)

(21) Appl. No.: 16/083,891

(22) PCT Filed: Mar. 24, 2017
(86) PCT No.: PCT/IB2017/051705

§ 371 Date: Sep. 11, 2018

(30) Foreign Application Priority Data
Mar. 29, 2016 (IN) 201641010908

(51) Int. Cl. C07K 1/18; C07K 1/20; C07K 1/22; C07K 1/36

(52) U.S. Cl. CPC C07K 1/18; C07K 1/20; C07K 2319/30; C07K 1/36; C07K 1/22

(57) ABSTRACT

The present invention discloses a process for purification of Fc fusion proteins using a series of steps. The process results in a final product that meets desired specifications for aggregates, unfolded proteins, glycosylation and purity, starting from broth containing high level of process and product related impurities. The fusion proteins are captured by Protein A resin, their isoforms separated by anion exchange chromatography and the undesired clipped, unfolded forms and aggregates separated by HIC using PPG resin. This protein is either directly formulated or further polished by cation exchange chromatography before the final formulation. The process is equally applicable for all fusion proteins containing an Fc portion and results in a product meeting all specifications for use as a biotherapeutic or a biosimilar.

### FIELD OF THE INVENTION

The present invention relates to a process for purification of Fc fusion proteins through a series of steps resulting in a final product that meets desired specifications for purity, aggregates, unfolded proteins and glycosylation. The present invention describes a process starting from crude cell free supernatants containing high concentrations of host cell and product related impurities till a final purified product meeting desired specifications is obtained.

### BACKGROUND OF THE INVENTION

Fusion proteins or chimeric proteins are created through joining portions of different proteins. Most biotherapeutic fusion proteins are produced by fusing a part of proteins such as ligand-binding portion of cytokine or growth factors, extracellular domains of lymphocyte antigens, or toxin, to a fusion partner which stabilizes the molecule and provides an extended half-life to the final fusion product.

The Fc region of human Immunoglobulin G1 (IgG1) is a popular fusion partner, selected for its ability to extend the half-life of proteins to which they are fused. The presence of the Fc portion enables recycling of the fusion protein through the salvage neonatal FcRn receptor as well as protects the fusion protein from lysosomal degradation, thereby enhancing its half-life. The Fc portion of the fusion protein also interacts with Fc specific cell surface receptors, as well as to some proteins of the complement system. The Fc fusion could be either at the N terminus or the C terminus of the partner protein. Some of the Fc fusion proteins currently approved for use in treatment of various diseases include belatacept, abatacept, alefacept, rilonacept, romiplostim, aflibercept and etanercept.

The active form of the Fc fusion proteins are dimers with certain degree of glycosylation, with monomers, aggregates, clipped products, unfolded protein and inappropriately glycosylated molecules constituting product related impurities. During purification, host cell related impurities as well as product related impurities need to be removed, such that the final product is free of both. The final product further requires to contain the desired amount of glycosylation and the correct glycan profile.

Protein purification is a series of steps intended to isolate one or few proteins from a complex mixture of broth containing cells, tissues or whole organisms. Usually, the protein products are associated with high levels of impurities and hence effective processes are required to obtain a purified form of product. The steps used in the process of purification include column chromatography and filtration and are chosen to reduce the levels of impurities to acceptable levels, while ensuring highest possible yield and quality of the final product.

The purification of biotherapeutic proteins requires a stable and highly reproducible process that results in removal of all product and process related impurities to the extent that allows the purified product to be qualified according to ICH guidelines. Fusion proteins, which are made by combining two or more unrelated proteins, are especially difficult to purify. The process of production of the fusion proteins results in generation of a number of different species of product related impurities due to clipping, aggregation and degradation as well as differences in proportions of glycosylation from that desired in the final product. The present invention describes a process of purification of Fc fusion proteins that is applicable to all Fc fusion proteins.

### BRIEF DESCRIPTION OF THE DRAWINGS

FIG. 1 illustrates a flow chart for a process of purification of fusion proteins from cell free supernatant.

FIG. 2 illustrates the SDS-PAGE of fractions from Protein A capture cycle for a representative Fc-fusion protein.

FIG. 3 illustrates the SDS-PAGE of fractions from anion exchange chromatography for the representative Fc-fusion protein.

FIG. 4 illustrates different fractions of the Fc-fusion protein subjected to anion exchange chromatography loaded on Iso Electric Focusing (IEF) gel.

FIG. 5(a) illustrates the HIC HPLC of the standard representative fusion protein.

FIG. 5(b) illustrates the HIC HPLC of different fractions of the representative fusion protein separated on Polypropylene Glycol (PPG) resin.

FIG. 6 illustrates the HIC HPLC of the representative fusion protein pooled after separation on PPG resin.

FIG. 7 illustrates the separation of monomers and aggregates on PPG for the representative fusion protein.

FIG. 8 illustrates the HPLC profile of the eluent from cation exchange chromatography of the representative fusion protein on Size-Exclusion Chromatography (SEC-HPLC) and HIC-HPLC.

FIG. 9 illustrates Sodium Dodecyl Sulfate Polyacrylamide Gel Electrophoresis (SDS-PAGE) of the final purified product of the representative fusion protein.

FIG. 10 illustrates the sialic acid ratio of different fractions of anion exchange chromatography of the representative fusion protein.

FIG. 11 illustrates the specification and the results of analysis of the final purified product of the representative fusion protein.

### DETAILED DESCRIPTION OF THE INVENTION

In order to more clearly and concisely describe and point out the subject matter of the claimed invention, definitions are provided for specific terms, which are used in the following written description.

The term “Fusion Protein” refers to proteins formed through joining of parts or whole of two or more proteins.

The term “Protein Purification” refers to a series of processes intended to isolate one or a few proteins from a complex mixture, usually cells, tissues or whole organisms or fermentation broth.

The term “Anion Exchange Chromatography” refers to a form of ion-exchange chromatography that uses resins or packings with functional groups that separates anions.

The term “Cation Exchange Chromatography” refers to a form of ion-exchange chromatography that uses resins or packings with functional groups that separates cations.

The term “Protein A Chromatography” refers to capture of Fc containing proteins on resin containing Protein A as a ligand, based on affinity of the Fc portion of the protein to certain epitopes of Protein A.

The term “Hydrophobic Interaction Chromatography” refers to a form of chromatography that uses resins with functional groups that separate proteins on the basis of their hydrophobicity.

The term “SDS-PAGE” refers to a sodium dodecyl sulfate polyacrylamide gel electrophoresis (SDS-PAGE), a technique for separating proteins based on their ability to move within an electrical current, which is a function of the length of their polypeptide chains or of their molecular weight.

The present invention discloses a process for purification of Fc fusion proteins through a series of steps resulting in a final product which meets desired specifications for aggregates, unfolded proteins, glycosylation and glycan profile. The process is capable of obtaining the desired purity of the final product starting from a broth containing variable levels of product and process related impurities.

The present invention discloses a unique series of steps to achieve the purification of fusion proteins. The efficiency of each step and the quality of the purified product obtained in each step varies with the sequence of steps employed during purification. The process described is applicable for purification of any fusion protein containing Fc portion.

The present invention relates to the process for the purification of protein comprising
a) capturing of the fusion protein from cell free supernatant by Protein A chromatography;
b) subjecting the eluate of step (a) to anion exchange chromatography;
c) subjecting the eluate of step (b) to hydrophobic interaction chromatography
d) subjecting the eluate of step (c) to cation exchange chromatography; and
e) collecting the eluate to obtain the purified protein

Cation exchange chromatography is an optional step which may or may not be included, depending on the fusion protein being purified. When the cation exchange chromatography is not used, the protein obtained after hydrophobic interaction chromatography may be used for formulation of the final product. The embodiments described herein may optionally encompass any tangential flow filtration, concentration, diafiltration or ultrafiltration between the chromatographic steps. The embodiments described herein may further comprise one or more viral inactivation steps.

The purification according to the present invention utilizes at least three major chromatographic steps i.e affinity chromatography, anion exchange chromatography and hydrophobic interaction chromatography. The eluant from hydrophobic interaction chromatography may also be subjected to cation exchange chromatography for further polishing of the protein for certain fusion proteins. This step is not required for the purification of all fusion proteins.

The sequence of steps employed in the present invention results in highly purified fusion protein, with improved purity and efficacy in the yield of the final drug product.

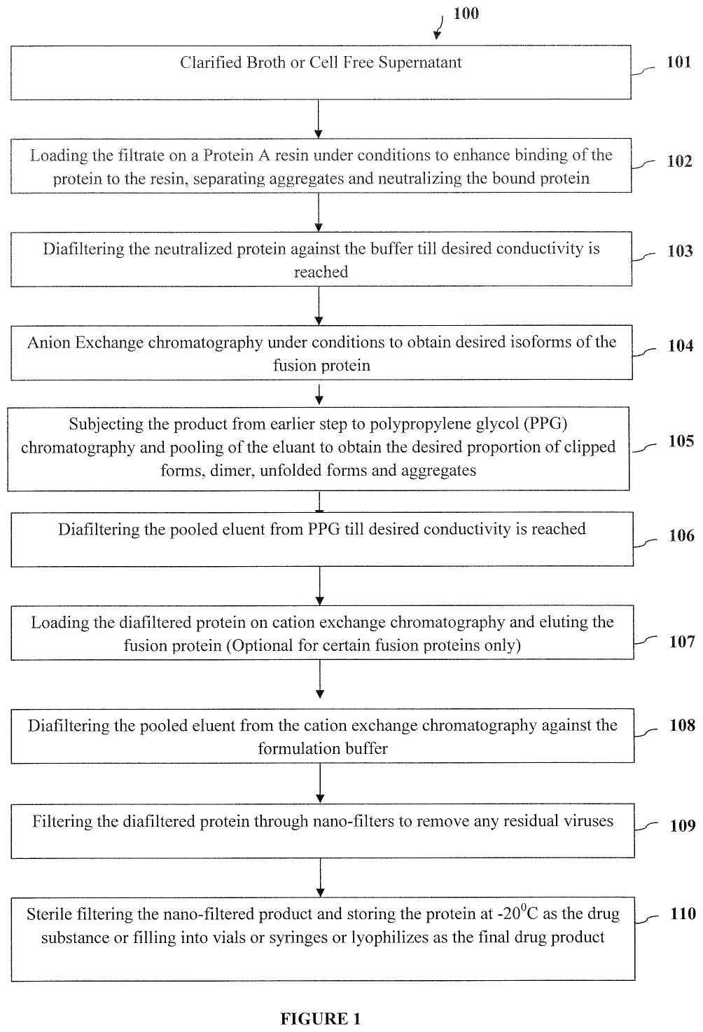

FIG. 1 illustrates a flow chart for a process of purification of fusion proteins from culture broth. The process (100) of purification starts with clarified broth (101). At step (102), the clarified broth is loaded on a Protein A resin under conditions that enhance the binding capacity of the resin. The unbound protein is removed in the flowthrough and by a series of washes. The bound protein is eluted under conditions that separate the aggregates from the dimers. The protein is held at low pH for viral inactivation and neutralized with alkali. At step (103), the neutralized protein is diafiltered till desired conductivity is reached. At step (104), the diafiltered protein is loaded on anion exchange resin under conditions that prevent binding of lower isoforms of the fusion protein. All the undesired isoforms and degradation products are further removed by a series of washes in this step. At step (105), the eluent is subjected to polypropylene glycol (PPG) chromatography, which separates the different forms of the protein such as the clipped product, unfolded protein, monomers, aggregates and dimers. The desired forms are pooled in this step. At step (106), the pooled sample is diafiltered till desired conductivity is reached. At step (107), the diafiltered protein is subjected to cation exchange, chromatography and the fusion protein is eluted. The fusion protein is eluted at high concentrations and exhibits desired specifications. At step (108), the pooled eluent from the cation exchange chromatography is diafiltered against the formulation buffer. At step (109), the diafiltered protein is filtered through nano-filters to remove any residual viruses. At step (110), the nano-filtered product is subjected to terminal sterile filtration and the protein is stored at −20° C. as the drug substance or filled into vials or syringes or lyophilized as the final drug product.

The final fusion protein thus obtained exhibits high purity.

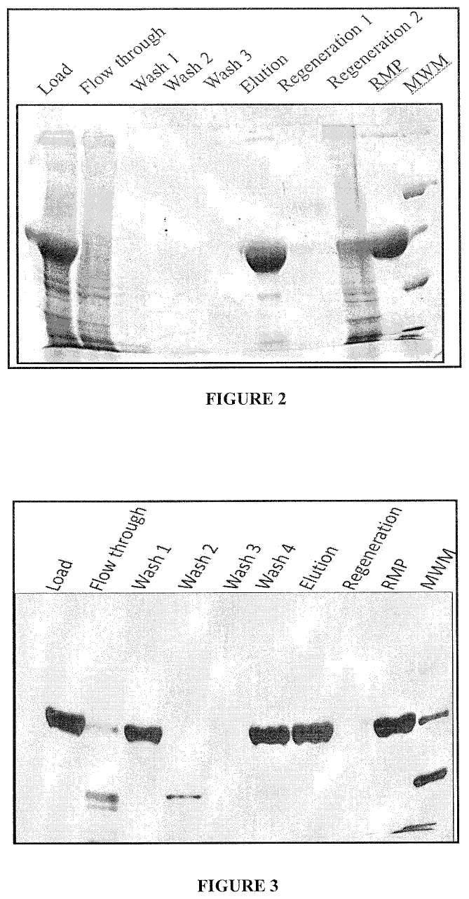

FIG. 2 illustrates the Sodium Dodecyl Sulfate Polyacrylamide Gel Electrophoresis (SDS PAGE) of fractions from Protein A capture cycle of a representative Fc-fusion protein. The cell free supernatant is loaded on a Protein A resin under conditions to enhance the binding capacity of the resin while also separating the aggregates from the desired dimeric form. 10% SDS-PAGE gel is loaded with the different fractions under reducing conditions. RMP (The standard for the fusion protein) and MWM (molecular weight marker) are also loaded as control. The gel is silver stained, which shows the presence of Fc fusion protein.

FIG. 3 illustrates the SDS-PAGE of fractions from anion exchange chromatography for the representative Fc-fusion protein. The fusion protein captured by Protein A resin is either diafiltered or diluted and loaded on an anion exchange resin under conditions that results in removal of undesired isoforms and degradation products. The different fraction from the cycle, RMP (The standard for the fusion protein) and MWM (molecular weight marker) are loaded on SDS-PAGE gel under reducing conditions. The gel is silver stained, which shows the removal of the degradation products and recovery of desired fusion protein in different fractions of anion exchange chromatography.

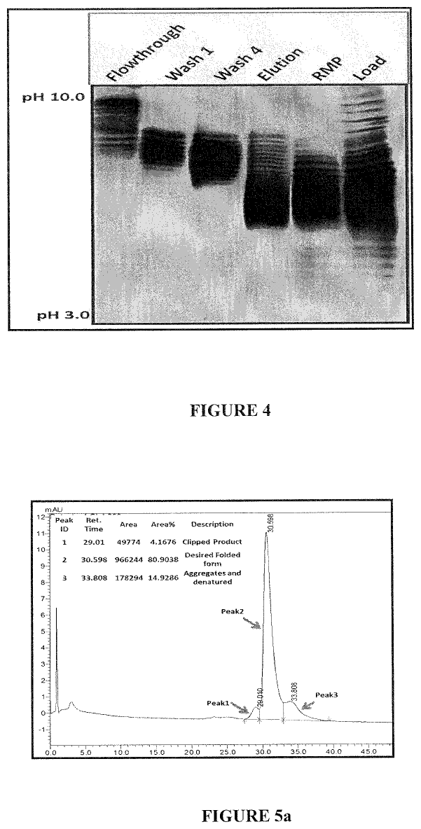

FIG. 4 illustrates 5 microgram of different fractions of the representative Fc-fusion protein subjected to anion exchange chromatography loaded on Isoelectric focussing gel (IEF) gel under denaturing conditions. RMP (The standard for the fusion protein) is also loaded as control. The gel is silver stained and shows the separation of different isoforms in different fractions.

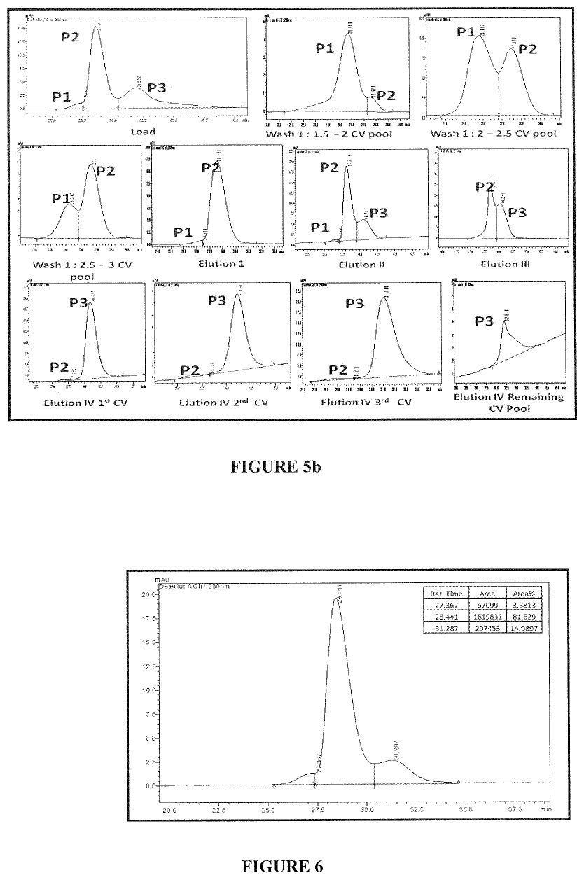

FIG. 5 (a) illustrates the HIC HPLC of the standard for the representative fusion protein. The chromatogram shows the separation of different forms of the fusion protein. Peak 1 consists of the clipped product, desired product is seen in Peak 2 and unfolded protein and aggregates are seen in Peak 3.

FIG. 5 (b) illustrates the HIC HPLC of different fractions of the representative fusion protein separated on PPG. The chromatogram shows the separation of different forms of the fusion protein in different fractions of the chromatographic run. Peak 1 consists of the clipped product, desired product is seen in Peak 2 and unfolded protein and aggregates are seen in Peak 3.

FIG. 6 illustrates the HIC HPLC of the representative fusion protein pooled after PPG chromatography. The results show the final pool having desired proportion of protein as depicted in Peak 1, 2 and 3.

FIG. 7 illustrates the separation of monomers, aggregates and dimers of the representative fusion protein on PPG resin. The different fractions obtained on chromatography injected on SEC-HPLC column show the clear separation of monomers, dimers and aggregates in the different fractions.

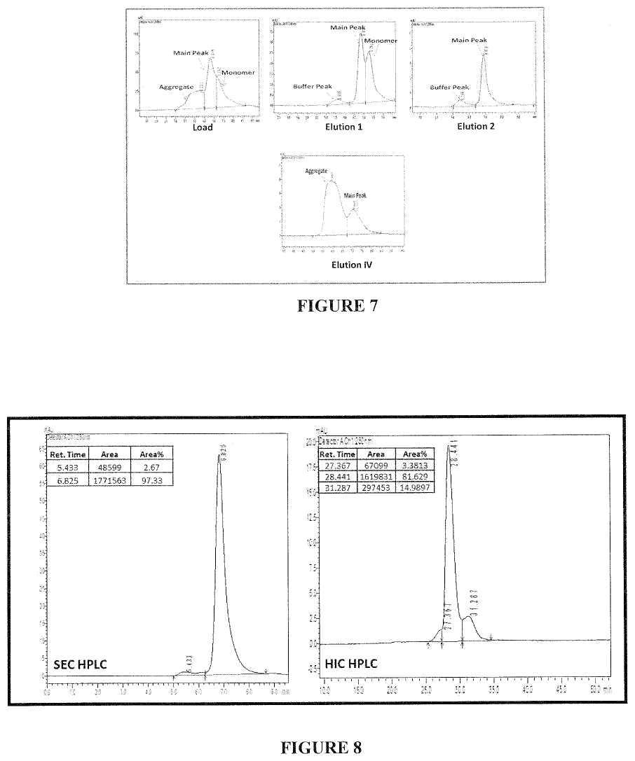

FIG. 8 illustrates the HPLC profile of the representative fusion protein eluted from cation exchange chromatography on SEC HPLC and HIC-HPLC. 25 μg of the eluent from cation exchange chromatography is injected into butyl HIC and SEC HPLC respectively. The results show the proportion of different peaks of fusion protein on Analytical butyl HIC column and proportion of aggregates on SEC HPLC.

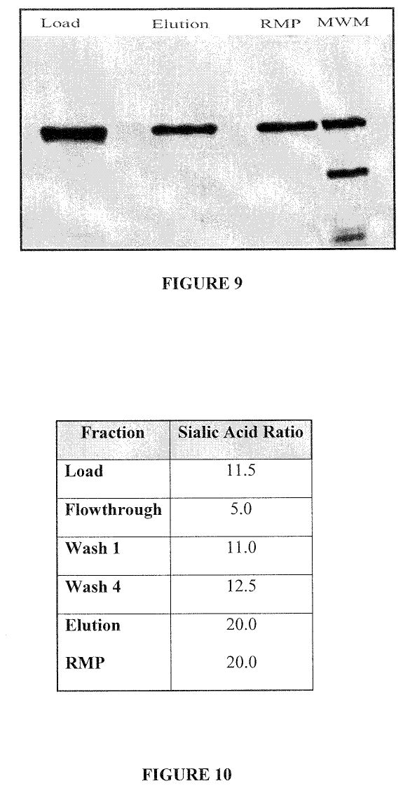

FIG. 9 illustrates SDS-PAGE of the final purified product of the representative fusion protein. The eluent after cation exchange chromatography is loaded on the SDS-PAGE. RMP (The standard for the fusion protein) and MWM (molecular weight marker) are also loaded as control. The gel is silver stained. The results illustrate the purity of the fusion protein.

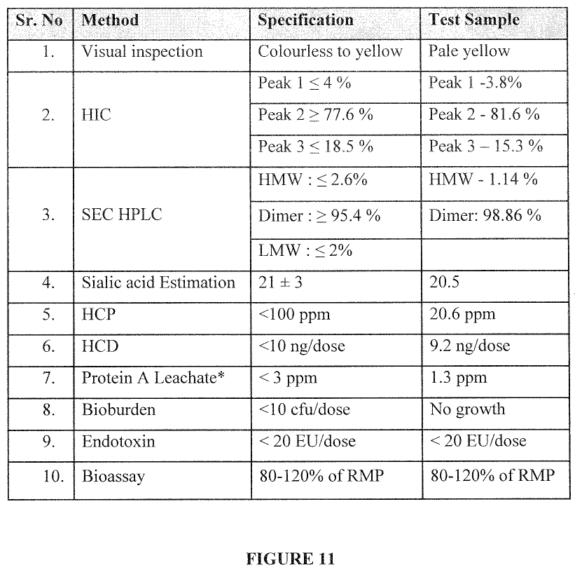

FIG. 10 illustrates the sialic acid ratio of different fractions of the representative fusion protein subjected to anion exchange chromatography. The lower isoforms are seen in the flow through and washes while the desired ratio is achieved in the elution fraction. RMP indicates the standard for the representative fusion protein.

FIG. 11 illustrates the specification and the results of analysis of the final product for the representative fusion protein obtained at the end of purification process. The results are tabulated as depicted in FIG. 11.

In order that this invention be more fully understood, the following preparative and testing examples are set forth. These examples are for the purpose of illustration only and are not to be construed as limiting the scope of the invention in any way.

### Example 1: Clarification

Cell free supernatant is generated from the fermentation broth by any one of the following methods:
Perfusates obtained from perfusion based fermentation are free of cells
Clarification by hollow fibre filtration of the broth containing cells
Clarification by depth filtration
Centrifugation followed by microfiltration using 0.1, 0.2 or 0.4 micron filters.

The final cell free supernatant obtained by the above method/s is free of particulate matter and cell debris and exhibits a turbidity of less than 10 NTU.

### Example 2: Protein A Chromatography

The cell free supernatant is loaded on a Protein A resin selected from Repligen Protein A, Poros Protein A, MabSelect Sure, Eshmuno Protein A or any other protein A resin. The Fc containing fusion protein binds to the Protein A resin while the unbound protein is removed in the flow-through as well as by a series of washes. The fusion protein is eluted with a pH gradient which separates the dimer from aggregates. The protein is held at low pH for 1 hour for the purpose of viral inactivation. The sample is then neutralized with alkali. This step results in the yield of 90-98% of the fusion protein.

A summary of the conditions used for of the representative fusion protein on Eshmuno protein A resin is given below:

| Step | Buffer | No. of CVs | Purpose |
|---|---|---|---|
| Equilibration | 25 mM Sodium phosphate | 3 | To prepare the column for binding |
| | buffer, pH 7.0 + 270 mM | | to Fc protein under the conditions |
| | NaCl + 10 mM EDTA | | of the sample load |
| Loading | Cell Free supernatant | — | Binding to Fc fusion Protein to the |
| | | | resin |
| Wash I | 25 mM Sodium phosphate | 3 | To remove HCP and other non- |
| | buffer, pH 7.0 + 270 mM | | specifically bound proteins |
| | NaCl + 10 mM EDTA | | |
| Wash II | 25 mM Sodium phosphate | 5 | |
| | buffer, pH 7.0 + 0.5M | | |
| | Arginine | | |
| Wash III | 25 mM Sodium phosphate | 10 | |
| | buffer, pH 7.0 + 1.5M | | |
| | NaCl + 5% IPA | | |
| Wash IV | 100 mM Sodium Citrate, | 5 | To remove IPA present in previous |
| | pH 6.0 | | wash |
| Elution I | A: 100 mM Sodium | 12 | To elute the Fc fusion protein |
| | citrate, pH 6.0 | | |
| | B: 100 mM Sodium | | |
| | citrate, pH 3.4 | | |
| | 0-100% B over 100 CV's | | |
| | 100% B over 2 CV's | | |
| Regeneration | 100 mM Sodium Citrate, | 2 | Removal of aggregates of Fc fusion |
| | pH 3.0 | | protein and other impurities bound |
| | | | to resin |

The neutralized protein is either concentrated and diafiltered or diluted to the desired conductivity before loading on anion exchange column.

### Example 3: Anion Exchange Chromatography

The protein is loaded on a column packed with anion exchange resin under conditions wherein the lower isoforms of the protein are unable to bind to the resin. The column is further washed to remove the lower isoforms and degradation products and eluted under conditions to recover only the desired isoforms of the fusion protein. All the lower isoforms and degradation products are removed in this step. The anion exchange resin used in this step could be from the group of DEAE (Diethylaminoethyl), ANX, EDA or Q.

The conditions used for a representative fusion protein separated onto DEAE-Sepharose FF are summarized in the table below:

| Step | Buffer Composition | No. of CVs | Purpose |
|---|---|---|---|
| Equilibration | 20 mM Sodium phosphate | 10 | To prepare the column for binding to the |
| | buffer, pH 5.8 + 25 mM | | representative fusion protein |
| | NaCl | | |
| Loading | Diafiltered sample from | — | Binding under conditions for removal of |
| | previous step | | lower isoforms in flow through |
| Wash I | Equilibration buffer | 5 | To remove HCP's bound non- |
| Wash II | 20 mM Sodium phosphate | 5 | specifically to resin |
| | buffer, pH | | |
| | 5.8 + 0.5% Tween80 | | |
| Wash III | 20 mM Sodium phosphate | 10 | |
| | buffer, pH 6.0 | | |
| Wash IV | 20 mM Sodium phosphate | 5 | To remove Tween and achieve a pH of |
| | buffer, pH 6.0 + 70 mM | | 6.0 on column |
| | NaCl | | |
| Elution | 20 mM Sodium phosphate | 5 | To elute the desired protein |
| | buffer, pH 6.0 + 250 mM | | |
| | NaCl | | |
| Regeneration I | 1M NaCl | 2 | Removal of impurities bound to resin |

The pH and conductivity of the salt used in the load and washes may be modified for different Fc Fusion proteins.

### Example 4: Polypropylene Glycol Chromatography

The eluent after anion exchange chromatography is subjected to hydrophobic interaction chromatography using PPG, a resin with different selectivity to the other HIC resins. The hydrophobic interaction chromatography separates aggregates, clipped products, monomers, dimers and unfolded proteins.

The eluent from the earlier step is prepared such that the protein binds completely to the PPG resin. The column is washed to remove undesired forms of the fusion protein. A series of elutions with decreasing concentration of salts is carried out to separate the different forms of the fusion protein. The different forms are estimated in each elution fraction and pooled such that the final pool meets the specification for the fusion protein.

The conditions used for the separation of different forms of a representative fusion protein on PPG resin are summarized below:

| Step | Buffer | No. of CVs | Purpose |
|---|---|---|---|
| Equilibration | 20 mM Sodium | 3 | To prepare the column for |
| | phosphate buffer pH | | binding of fusion protein |
| | 7.0 + 430 mM Sodium | | |
| | citrate + 120 mM NaCl | | |
| Loading | Eluant from earlier step | — | For binding of the fusion protein |
| | | | to column |
| Wash I | 20 mM Sodium | 3 | For removal of clipped products |
| | phosphate buffer pH | | (Peak1) |
| | 7.0 + 430 mM Sodium | | |
| | citrate + 120 mM NaCl | | |
| Elution I | 20 mM Sodium | 5 | For separation of clipped product |
| | phosphate buffer pH | | and monomers |
| | 7.0 + 390 mM Sodium | | |
| | citrate + 120 mM NaCl | | |
| Elution II | 20 mM Sodium | 3 | Elution dimer |
| | phosphate buffer pH | | |
| | 7.0 + 380 mM Sodium | | |
| | citrate + 120 mM NaCl | | |
| Elution III | 20 mM Sodium | 5 | For separation of dimer from |
| | phosphate buffer pH | | unfolded protein and aggregates |
| | 7.0 + 350 mM Sodium | | |
| | citrate + 120 mM NaCl | | |
| Elution IV | Gradient | 17.5 | Separation of dimers, unfolded |
| | A: 20 mM Sodium | | proteins and aggregates |
| | phosphate buffer pH | | |
| | 7.0 + 350 mM Sodium | | |
| | citrate + 120 mM NaCl | | |
| | B: 20 mM Phosphate | | |
| | buffer pH 7.0 | | |
| | 0-100% B over 17.5 CV's | | |
| | 100% B over 2 CV's | 2 | |

The pH and salt concentrations used for the separation could vary slightly for different fusion proteins, although the general strategy for the separation remains the same.

Most fusion proteins can be directly diafiltered and formulated after this step. At the end of this step, the product related impurities have been completely removed while HCP, HCD and Protein A leachates are in the acceptable range. Glycosylation and glycan profile are also as desired.

However, some fusion proteins may require a further polishing step for removal of traces of process and product related impurities. A cation exchange chromatography is carried out for such polishing to remove traces of HCP, HCD, Protein A leachate, aggregates and other impurities.

### Example-5: Cation Exchange Chromatography

The eluent pooled from the PPG chromatography step is diafiltered till the conductivity reaches 2-3 mS/cm.

The diafiltered protein is loaded on cation exchange resin under conditions optimized to allow maximum binding capacity. A cation exchange resin that has a high binding capacity like Gigacap S650 or similar resins is chosen for this step. Up to 70 mg of protein is bound per ml of the resin in this step. The column is washed under conditions that remove HCP, HCD and Protein A leachates. The protein is eluted under conditions to separate aggregates and lower isoforms. The fusion protein is eluted at concs of >20 mg/ml with <3% aggregates and exhibits the final desired specification.

The steps used in the cycle for a representative protein are summarized below:

| Step | Buffer | No. of CVs | Purpose |
|---|---|---|---|
| Equilibration | 25 mM Sodium | 3 | To prepare the column for binding of |
| | acetate buffer pH | | protein |
| | 4.3 + 50 mM NaCl | | |
| Loading | Diafiltered after HIC | — | Binding of desired protein while HCP |
| | | | and HCD flows through |
| Wash I | Equilibration buffer | 3 | To remove unbound proteins |
| Wash II | 20 mM Sodium | 3 | To remove Sodium acetate from |
| | phosphate buffer, | | previous wash and bring it to |
| | pH | | phosphate buffer |
| | 5.8 + 0.5% Tween80 | | |
| Elution I | 20 mM Sodium | 5 | To elute the desired protein |
| | phosphate buffer, | | |
| | pH 6.0 + 250 mM | | |
| | NaCl | | |
| Regeneration I | 1M NaCl | 3 | To remove protein bound to the resin |

### Example 6: Final Formulation

The eluent pooled from the cation exchange chromatography step is diafiltered against the formulation buffer, subjected to nano filtration followed by terminal sterile filtration and stored as Drug Substance.

The purification process described in the present invention with minor modification and deletions of one or more steps is equally applicable for all Fc containing fusion proteins. The sequence of the steps can also be interchanged without affecting the final output. The process results in obtaining purified protein that meets the specifications for use as a biotherapeutic or a biosimilar.

1) A process for the purification of protein comprising the steps of
a) capture of the protein from broth using Protein A resin;
b) subjecting the eluate of step (a) to anion exchange chromatography;
c) subjecting the eluate of step (b) to hydrophobic interaction chromatography;
d) subjecting the eluate of step (c) to cation exchange chromatography; and
e) collecting the eluate to obtain the purified protein with optional steps of diafiltration being carried out between chromatographic steps.

2) A process according to claim 1, wherein the protein being purified could be any Fc Fusion Protein including belatacept, abatacept, alefacept, rilonacept, romiplostim, aflibercept or etanercept.

3) A process according to claim 1 where the fusion protein can be purified by excluding cation exchange chromatography at the end of the first three steps.

4) A process according to claim 1, wherein the resin used for capture could be any resin with Protein A ligand attached to it—including Repligen Protein A, Eshmuno Protein A, MAbSelect Sure, MAbSelect Extra and others.

5) A process according to claim 1, wherein the Protein A column is washed with 0.5-1M arginine for removal of Host Cell related impurities.

6) A process according to claim 1, wherein the Protein A column is washed with 1.5 M NaCl and 5% IPA for removal of Host Cell related impurities.

7) A process according to claim 1, wherein the elution of the protein from Protein A is carried out with a gradient optimized for separation of aggregates.

8) A process according to claim 1, wherein the elution of the protein from Protein A is carried out with a gradient from pH 7 to 3 or from pH 6 to 3 or from pH 5 to 3 or with a gradient starting from a pH 5-7 till pH 3.6-3.0, optimized to separate aggregates from the dimers during elution such that <5% aggregates are eluted out even when the load contains up to 30% aggregates.

9) A process according to claim 1, wherein isoform separation is carried out with an anion exchange resin from within the group of DEAE, Q, EDA or any other anion exchanger.

10) A process according to claim 1 where the protein is loaded on the anion exchanger at a conductivity that prevents binding of lower isoforms, thereby causing the lower isoforms to be removed in the flowthrough.

11) A process according to claim 1 where the conductivity of loading is chosen from between 5-10 mS/cm to prevent binding of lower isoforms to the anion exchange resin.

12) A process according to claim 1 where lower isoforms are further removed by washing the anion exchange column with salts at a concentration that elutes undesired isoforms of the protein.

13) A process according to claim 1 where lower isoforms are washed off from the anion exchange column with NaCl at a concentration of 50-100 mM.

14) A process according to claim 1 where separation of aggregates, clipped products, unfolded proteins, monomers and dimers is carried out by Hydrophobic interaction chromatography.

15) A process according to claim 1 where the HIC resin is Polypropylene glycol.

16) A process according to claim 1 where the separation on PPG resin is carried out with a series of elutions with buffer containing reducing concentrations of salt followed by pooling of the fractions from the different elutions in a manner as to obtain a final product with the desired ratio of dimers, unfolded protein, clipped products and aggregates.

17) (canceled)

18) (canceled)

19) A process according to claim 1 where Cation Exchanger chosen is Gigacap S-650.

20) A process according to claim 1 where 50-70 mg of protein is bound per ml of the cation exchange resin.

21) A process according to claim 1 where the fusion protein is eluted from cation exchange column with a buffer without addition of salts.

22) A process according to claim 1 where the concentration of protein in the eluant from cation exchange chromatography is >20 mg/ml.

23) (canceled)
